(19) United States
(12) Patent Application Publication
(10) Pub. No.: US 2009/0209803 A1
(43) Pub. Date: Aug. 20, 2009

(54) Adjuvant brachytherapy apparatus and method for use with kyphoplasty

(76) Inventor: Paul A. Lovoi, Saratoga, CA (US)

Correspondence Address: Thomas M. Freiburger, P.O. Box 1026, Tiburon, CA, 94920, US

(21) Appl. No.: 12/070,176

(22) Filed: Feb. 14, 2008

(51) Int. Cl. A61M 36/06

(52) U.S. Cl. 600/4

(57) ABSTRACT

In a kyphoplasty procedure to expand and repair a damaged vertebra, diseased bone around the vertebral fraction zone is irradiated by use of a small radiation source inserted through the cannula used in the kyphoplasty procedure.

### BACKGROUND OF THE INVENTION

This invention pertains to administration of brachytherapy following treatment of spinal compression fractures which often result from the weakening effects of metastatic cancer within the vertebrae, especially in breast, prostate and lung cancer patients.

Vertebral compression fractures are painful due to distortion of the spinal cord, often resulting in loss of mobility and/or motor control, and require palliative or curative treatment. Traditionally, such treatment comprised external beam radiotherapy, often in conjunction with corticosteroids. External beam radiation can be complicated, however. The radiation directed to the body of the vertebra in question must pass through any overlying anatomy, and the nearby spinal cord is particularly sensitive to radiation. A study (reported in Lancet, Aug. 20-26, 2005: 366(9486): 643-648) showed that surgery followed by radiation is more effective, however, and in addition provides immediate pain relief. Such surgery preferably reduces spinal deformity and stabilizes the spine.

One minimally invasive surgical procedure used in this regard is kyphoplasty in which a cannula is placed into the patient's back lateral of the spinous process and advanced adjacent the spinal foramen into the body of the affected vertebra. A balloon or other expandable member is next passed into the vertebral body and inflated to reduce spinal deformity. Following balloon removal, a cementitious material is injected into the space created by the balloon, and allowed to cure. Such treatment is customarily bilateral, proceeding from both the left and right sides of the spinous process, giving immediate relief to many patients, and restoring or tending to restore mobility and motor control. In addition to the study findings mentioned, it has also been established that intracavitary brachytherapy is preferable to external beam therapy in that it is more sparing of normal tissue. Since it emanates from within the cavity created by the previous procedure, it is focused on the immediately adjacent tissue where any diffuse disease is likely situated. The radiation need not pass through the overlying anatomy in order to reach the target tissue. From the above, it is clear that a protocol combining minimally invasive surgery and brachytherapy would greatly benefit patients' suffering from vertebral compressive fractures.

### SUMMARY OF THE INVENTION

The method of this invention comprises surgery to reduce spinal deformity resulting from compressive vertebral fracture, (for reasons of disease, old-age, injury, etc.), followed by adjuvant brachytherapy and then stabilization of the vertebra. The preferred surgery is kyphoplasty wherein a balloon is used to realign the spinal deformation and where a bone cement, for example a polymethylmethacrylate material (Kyphon, Inc., 1221 Crossman Ave., Sunnyvale, Calif. 94089) is used to preserve the realignment after surgery.

After spinal realignment, a cavity remains between or within the structure of the bone which has been forcibly reconfigured. In the method of the invention, a radiation source is positioned within this cavity, and radiation delivered to the adjacent bone thought potentially to host proliferative disease cells which could initiate recurrence of further symptoms. The radiation may be delivered from within a balloon or directly to the tissue without a balloon, and is shielded or otherwise controlled in a manner avoiding irradiation of the spinal cord. Equally, measures can be taken to manipulate the source within the cavity to achieve the prescribed radiation dose in an optimal manner. Radiation sensors, for example MOSFET type, may be positioned to monitor absorbed dose. These may be skin mounted, or advanced percutaneously on needles and positioned to warn of overdose. The sensors can also be used in treatment planning or to verify dose delivered.

Optionally, a radiosensitizer can be infused or applied within the cavity in a manner facilitating the prescribed therapeutic effect, but with a lower absolute dose of radiation than otherwise would be possible. Delivery of the agent can be from the surface of the kyphoplasty balloon used to reduce spinal deformity, or on a later treatment balloon in a manner as disclosed in U.S. Pat. No. 7,018,371. It can also be swabbed in the cavity surface or as a wash, subsequently aspirated where such method is carried out through the cannula. As will be apparent from the discussion below, it is preferred that the cavity from within which the radiation is emitted be filled with an attenuating fluid. If the fluid, preferably saline, is injected directly into the cavity created by realignment, the fluid can advantageously comprise the radiosensitizer. Alternatively, a balloon applicator can be used to contain the attenuating medium, and if the balloon membrane is porous, the fluid can again comprise the radiosensitizer and be diffused from within. In either case, the radiation source is operated from within this fluid. A typical balloon applicator is described in U.S. Pat. No. 6,413,204 and is further described below. Such apparatus is well known to those of skill in the art.

After delivery of the prescribed dose of radiation (including any administration of a radiosensitizer) the fluid is drained or aspirated and the radiation apparatus (including any applicator) is withdrawn and the bone cement injected through the cannula into the vertebral cavity and cured or allowed to set in order to preserve the realigned spinal configuration. The cannula is subsequently withdrawn.

The preferred radiation sources of this invention are miniature x-ray sources constructed, for example, in keeping with the principles described in Atoms, Radiation and Radiation Protection, Second Edition, John E. Turner, Ph.D., CHP, 1995, John Wiley & Sons, Section 2.10. Such a source can emit isotropically and be shielded so as to protect at-risk anatomical structures (e.g., the spinal cord), or it can be directional (only emitting through a predetermined solid angle) and manipulated so as not to expose sensitive anatomy—particularly the dura matter and spinal cord. Shielding of isotropic x-ray sources to achieve similar directional effects is discussed in co-pending U.S. patent application Ser. Nos. 11/471,013 and 11/471,277. Isotope sources in principle can be used similarly to x-ray tubes; however, their use is complicated by the isotropic nature of their emissions, the fact that they can't be turned on and off or modulated in the manner of x-ray tubes, and the fact that their radiation spectrum requires extensive safety measures be taken to protect attending personnel. Miniature x-ray sources allow radiotherapy to be delivered in virtually any medical facility, not only from within the bunkers that are necessary to house isotope sources or external beam units, and which for economic reasons are located only in major population centers.

Because of the small scale of cavities formed by spinal realignment, measures may need to be taken to moderate the absorbed dose in the realignment cavity surfaces to avoid necrosis of normal tissue. Conventional hardening of the x-ray source may be used, or the methods described in U.S. patent application Ser. No. 11/925,200 can be employed to control and/or moderate the surface dose without detracting from delivery of the prescribed dose where desired. The disclosure of U.S. patent application Ser. No. 11/925,200 is incorporated herein in its entirety by reference.

By utilizing x-ray sources and practicing this invention, it is apparent that improved brachytherapy treatment results and can be made available to a much larger patient population than before.

### DESCRIPTION OF THE DRAWINGS

FIG. 1 is a side elevation view depicting a collapsed vertebra resulting from a compression fracture.

FIG. 2 depicts the vertebral compression fracture of FIG. 1 with a posteriorly placed cannula positioned obliquely into the body of the vertebra to access the fracture zone.

FIG. 3 is a top view of the subject vertebra depicted in FIGS. 1 and 2 with bilateral cannulae positioned as depicted in FIG. 2.

FIG. 4 is a side view depicting an expanded balloon as placed within the fracture zone and inflated, forming a cavity between upper and lower plates of the fractured vertebra.

FIG. 5A is a similar view showing a brachytherapy balloon applicator positioned within the realigned cavity filled with attenuating fluid, said balloon including an optional covering to deliver a radiosensitizer to the cavity surface.

FIG. 5B is similar to FIG. 5A, but shows the applicator balloon without the optional covering, and further shows a source within the balloon, mounted on a catheter or cable.

FIG. 5C is similar to FIGS. 5A and 5B, but uses the kyphoplasty balloon for brachytherapy rather than a separate applicator.

FIG. 6 is another side view showing a radiation source operating from within the cavity without a balloon.

FIG. 7 depicts the cavity in its realigned configuration being filled with cement to preserve spinal realignment.

FIG. 8 is a side view showing vertebra following after kyphoplasty and brachytherapy.

FIG. 9 is a perspective view showing a concentric, forwardly emitting directional x-ray source for use with the invention.

FIG. 10 depicts a side emitting x-ray source emitting through a solid angle from the axis of the source.

FIG. 11 is a schematic sectional side view showing a distal portion of a brachytherapy balloon applicator with a source guide fastened to both distal and proximal ends of the applicator balloon, for use in the method of the invention.

FIG. 12 is a schematic perspective view showing a manipulator suitable for manipulating a source in response to commands from a central controller to deliver a brachytherapy prescription.

### DESCRIPTION OF PREFERRED EMBODIMENTS

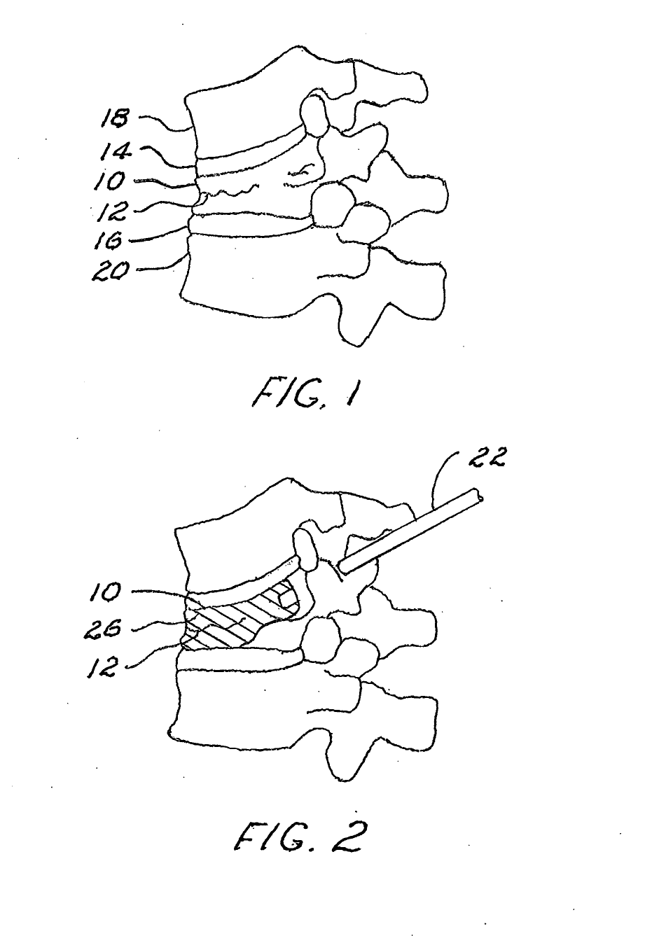

FIG. 1 is a lateral view of human spinal anatomy with a subject vertebra 10 exhibiting a compression fracture 12, in a patient to undergo a kyphoplasty procedure (See for example, American Academy of Orthopaedic Surgeons, 6300 North River Road, Rosemont, Ill. 60018, or search “Kyphoplasty” on website orthoinfo.aaos.org). The adjacent upper disc 14 and lower disc 16 are positioned respectively between upper vertebra 18 and lower vertebra 20. The anterior of the patient is to the left of the figure.

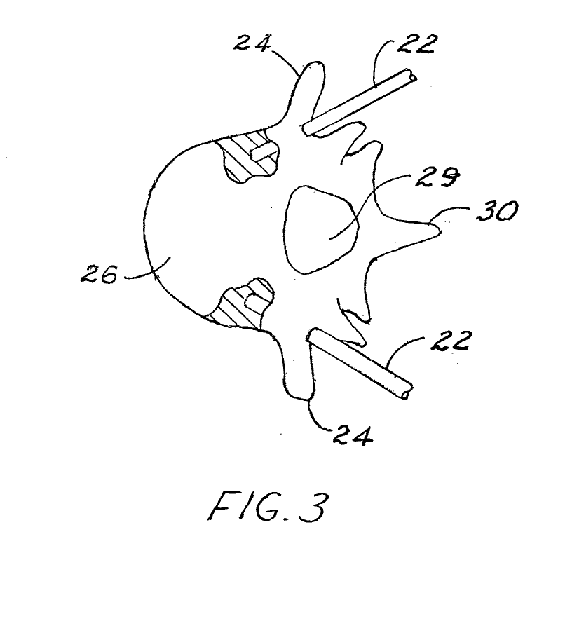

FIG. 2 is similar to FIG. 1; however, a cannula 22 is now positioned through the posterior skin of the patient (not shown), the transverse process 24 (see FIG. 3) of the vertebra 10 and into position to access the fracture zone 12 in the body 26 of the vertebra. In actuality, the cannula placement and, in fact the whole of the kyphoplasty procedure, including the brachytherapy of the invention, is generally bilateral.

FIG. 3 is a superior view of the vertebra 10 shown in FIG. 2. The cannulae 22 are placed from both sides of the spinal process 30, through each transverse process 24, passing so as to avoid the spinal foramen 29 and spinal cord and dura matter within (not shown), and onward into the body 26 of the vertebra 10. From this positioning, instrumentation (not shown) may be introduced through the cannula into the fracture zone (as shown in FIG. 2) to effect the intended kyphoplasty and brachytherapy.

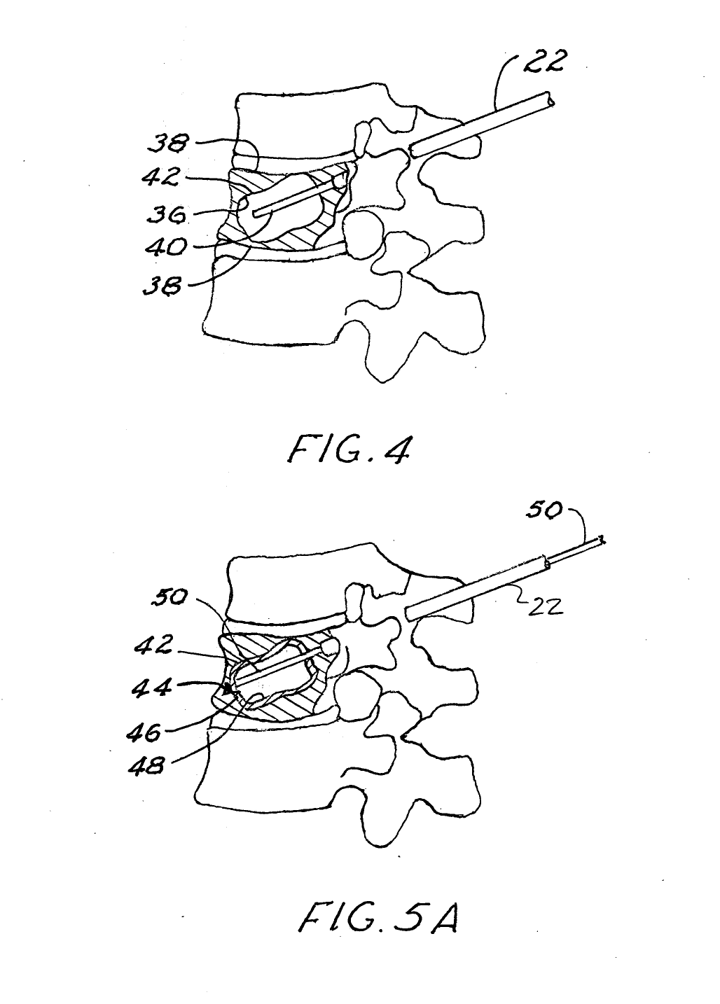

FIG. 4 is again a lateral view showing the fractured vertebra 10 having been realigned to a more normal anatomical configuration by inflation of a kyphoplasty balloon 36 which has been advanced on a shaft 40 through the cannula 22, into the fracture 12, and inflated. As may be seen, the upper and lower vertebral end plates 38 have been separated. On removal of the kyphoplasty balloon 36, a cavity 42 remains.

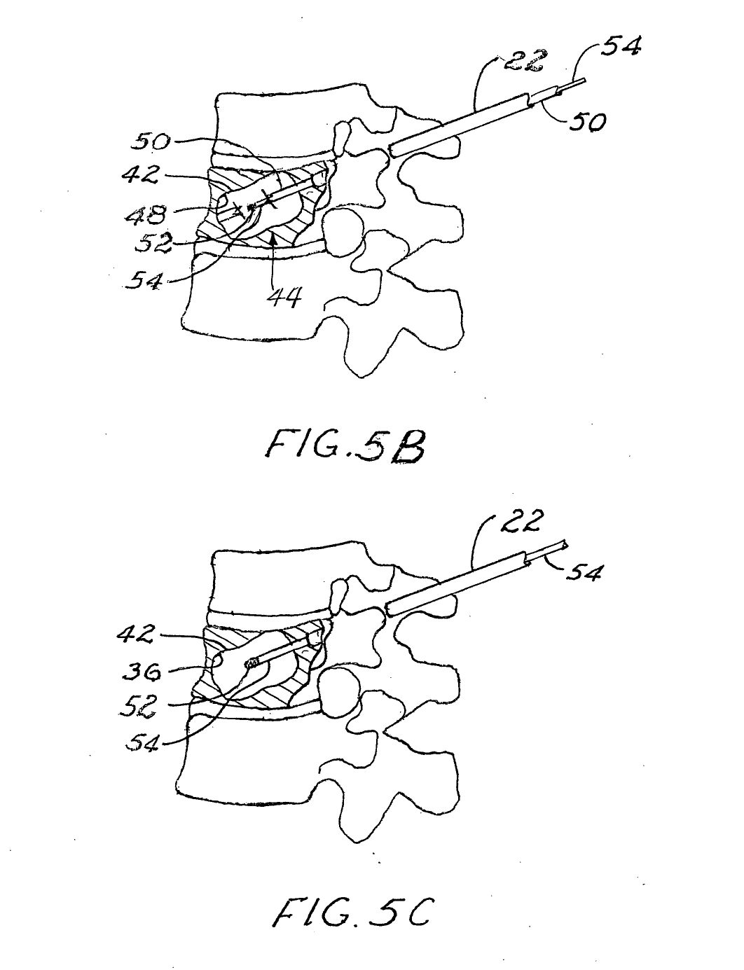

FIG. 5A shows a conventional brachytherapy balloon applicator 44 comprising a shaft-mounted balloon which, after removal of the kyphoplasty balloon, has been positioned within the cavity 42 and inflated with an attenuating fluid, preferably saline. A conventional hub at the proximal end of the applicator shaft with annulus sealing means, for example an O-rig, can be used for fluid control (neither shown). Also shown is an optional covering 46 on the external surface of the applicator balloon 48 which can be used to administer a radiosensitizing agent such as taxol, preferably in combination with any of misonadizole, metronidazole, etanidazole, 5-fluouracil, texaphrin, RSR13™, C225, cyclooxygenase-2 inhibitor, beta interferon, or a prodrug of any of the above, to the surfaces of the fracture cavity 42. Such methods and coverings are as described in co-pending U.S. patent application Ser. No. 11/639,495, incorporated herein by reference in its entirety. The agents can be absorbed into the covering 46 before introduction into the body, perfused through the balloon skin if it is permeable, or can be infused through the cannula 22 outside the shaft 50 of the applicator 44, and diffused into and through the covering 46. The radiosensitizing agent can be chosen to reduce the radiation dose necessary to achieve the desired therapeutic effect, whether that effect be palliative or curative. If desired, the sensitizer can be injected through the cannula 22 into the cavity 42 directly without a balloon, and aspirated after an appropriate time for agent migration into the cavity surfaces by capillary action or diffusion. Alternatively, the sensitizer may be swabbed onto the cavity surfaces through the cannula by conventional methods.

After the radiosensitizing agent has been applied as in FIG. 5A, a source on a catheter or cable is introduced into the balloon through the shaft 50 in a manner similar to that shown in FIGS. 5B or 5C.

FIG. 5B shows the apparatus of FIG. 5A without the optional balloon covering 46 of FIG. 5A, with a radiation source 52 mounted on a catheter or cable 54 inserted through the shaft 50 of the applicator 44. A conventional hub at the proximal end of the applicator shaft with annulus sealing means, for example an O-ring, can be used for fluid control (neither shown).

Note that in some circumstances, it is possible to eliminate a separate brachytherapy applicator and make use of the kyphoplasty balloon 36 (see FIG. 4) for containing the attenuating fluid and from which the radiotherapy can be delivered. Such a case is shown in FIG. 5C, where the attenuating fluid can optionally comprise the fluid used to realign the spinal anatomy (as shown in FIG. 4), and the radiation source 52 and its catheter or cable 54 can be introduced into the balloon 36 through the cannula 22. Again, a conventional hub with annulus sealing means (not shown), for example an O-ring, can be used for fluid control between the cannula 22 and the source catheter or cable 54.

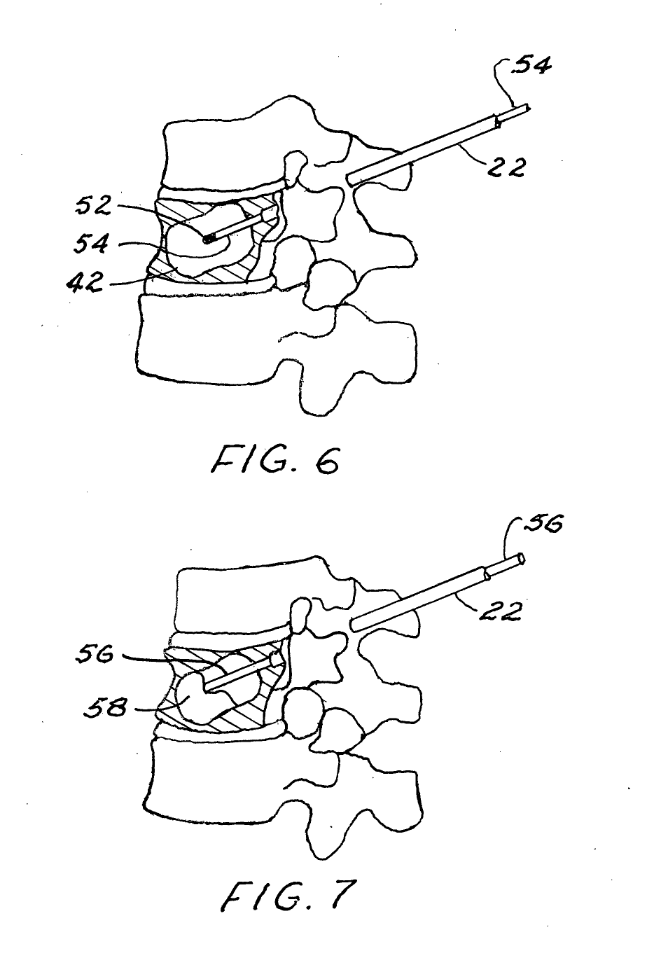

FIG. 6 shows a radiation source 52 mounted at the end of a catheter or cable 54, positioned within the cavity 42 and emitting therapeutic radiation to the cavity surfaces and into the diseased bone. With this method alternative, there is no brachytherapy applicator used, and the attenuating fluid, if used, is injected directly into the cavity through the annulus between the cannula 22 and the source cable 54. A conventional hub with annulus sealing means, for example an O-ring, can be used for fluid control (not shown). Direct injection of the fluid into the cavity also offers the opportunity to add the radiosensitizer to the attenuating fluid, thus eliminating the separate radiosensitizer administration step described in connection with FIG. 5.

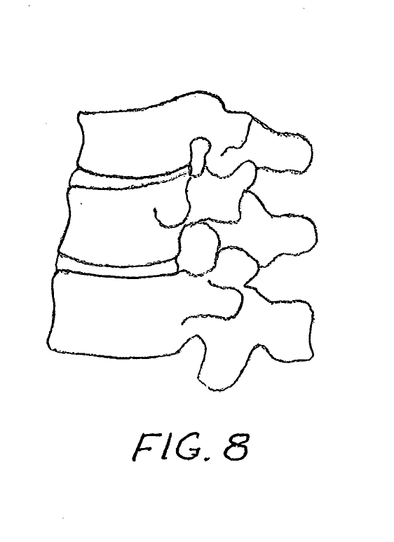

In FIG. 7, following vertebral realignment and brachytherapy, the balloon or cavity has been drained or aspirated, and any balloon, radiation source and catheter or cable have been removed from the cavity and replaced by an injection tube 56 for injecting cement 58 into the cavity to stabilize the realigned position of the fractured vertebra. The cement 58 is shown partially filling the cavity 42. After filling is complete, the injection tube is withdrawn, and the cement is allowed to set as necessary.

FIG. 8 shows the realigned spinal configuration after kyphoplasty and brachytherapy.

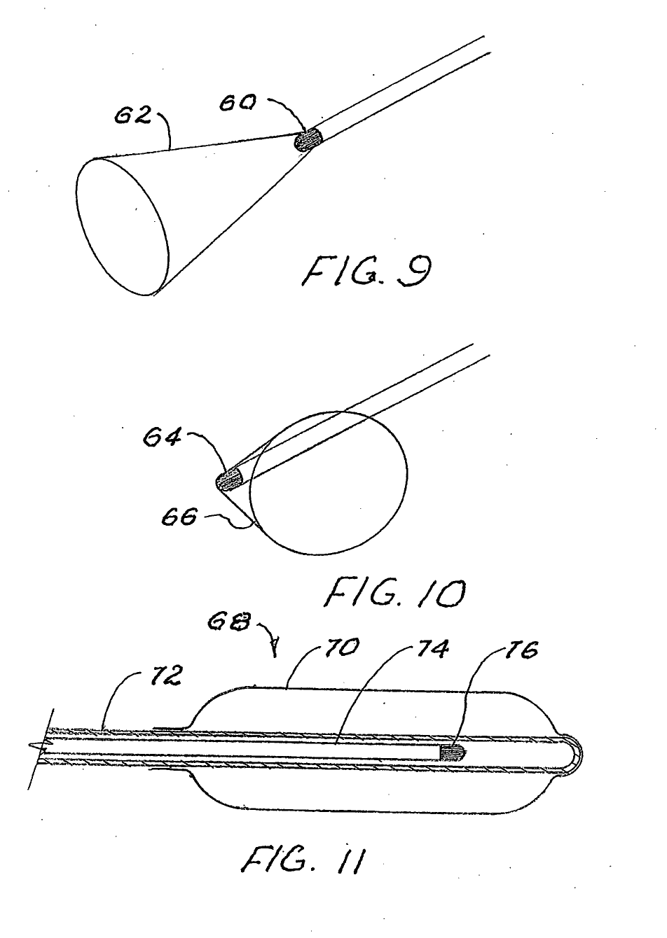

FIG. 9 shows in perspective an exemplary, forward-emitting radiation x-ray source 60 for use in the process of the invention. The geometry of the forwardly directed radiation cone or ellipsoid 62 can be engineered to suit the preferences of the radiation practitioner by x-ray tube target design. Electronic x-ray sources are commonly mounted on the end of a high-voltage cable which is manipulated within a source guide, cannula or other support structure within a balloon (see discussion in connection with FIG. 11), and manipulated in response to commands from a central controller programmed to optimize delivery of radiotherapy conforming to a predetermined prescription. In the case of a forward emitting source, the source 60 would not be withdrawn from the cavity to the extent that the radiation cone could intersect the spinal cord and its protective coverings. Such brachytherapy applicators are described in U.S. Pat. No. 6,413,204 and elsewhere. X-ray tubes of the type preferred often require cooling as well as electrical power, and such apparatus is described in U.S. Pat. No. 7,127,033.

FIG. 10 shows in perspective a similar x-ray source 64 as that in FIG. 9, but in this case the emissions are directed to the side, away from the axis of the x-ray tube, emitting throughout a predetermined solid angle 66. The sources in FIGS. 9 and 10 differ primarily in their target design. In this case, manipulation of the source 64 would preclude rotation and translation in a manner that would cause the spinal cord to be impacted.

FIG. 11 is a partial side view of the distal portion of a conventional brachytherapy balloon applicator 68. The balloon 70 is preferably fastened to a source guide 72 at both the distal and proximal ends of the balloon 70. Such two-point fixation is preferred in that it is more effective at positioning the source accurately within the balloon. However, double fixation is not necessary. An applicator source guide 72 fastened only at the proximal end of the balloon, and wherein the source is exposed to the cavity from within the balloon, may be used without departing from the scope of the invention. In either configuration, the source cable (or catheter) 74 is situated within the source guide 72, and in practice, is manipulated within the balloon 70 in response to a central controller programmed to deliver brachytherapy to a predetermined prescription. Such intracavitary brachytherapy is well known and the apparatus variations and methods disclosed herein will be thoroughly understood by those of skill in the art.

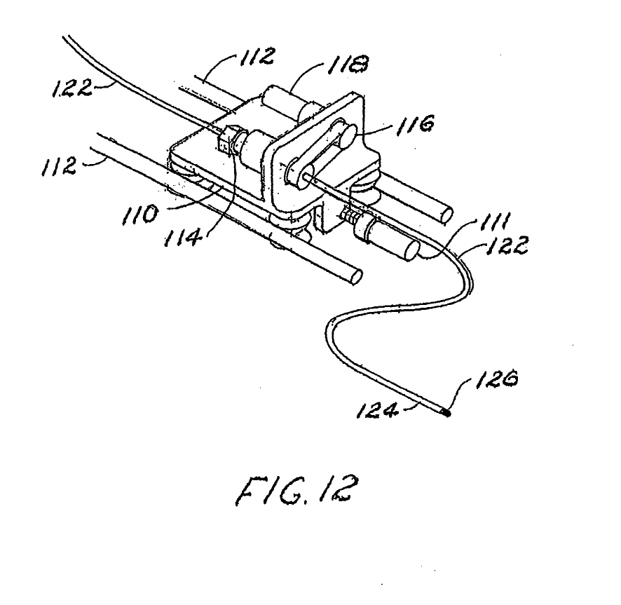

An exemplary source manipulation apparatus for use with sources of this invention is shown in schematic perspective in FIG. 12, and is capable of imparting translation and rotation to a source at the distal end of a catheter or cable apparatus in response to central controller input. A sled 110 is riding on and confined to rails 112, with its translation actuated by a servo-motor 111. A rotary spindle and collet 114 is mounted on the sled 110 in bearings (not shown), and connected by a belt or gear drive 116 to a servo-motor 118. On the distal end 124 of the cable or catheter 122 is mounted the source 126. The collet grips the catheter or cable 122 so that the source 126 moves with the spindle. The servos 111 and 118 are responsive to the central controller (not shown) which manages delivery of the treatment plan to prescription. The prescription and treatment plan are determined before radiotherapy, typically based on imaging of the apparatus within the anatomy by conventional x-ray or CT methods and the known dose required to achieve the desired therapeutic effect. Such planning is customarily by an automated process and will assure normal tissue, particularly the spinal cord, is protected from radiation as completely as possible. As explained earlier, sensors may be placed to assure safety during treatment and additionally, their output may be integrated into the central controller and thus into source manipulation.

Several variations in method steps and apparatus embodiments are suggested herein. Other combinations of elements may be used without departing from the scope of the invention. By utilizing brachytherapy in combination with kyphoplasty in accordance with the principles disclosed, many patients will find relief from pain, and others an outright cure for their disease. Due to the use of x-ray therapy, treatment venues will not be as limited as is presently the case.

I claim:

1. A method for administering radiation therapy along with a spinal vertebral kyphoplasty, comprising:
in the kyphoplasty procedure, following placement of at least one cannula obliquely into the body of a collapsed or damaged vertebra and inflation of a fracture zone with a fluid delivered via the cannula, inserting through the cannula an expandable brachytherapy balloon applicator within the fracture zone,
through a shaft of the balloon applicator, inflating a balloon of the applicator within the expanded fracture zone with an inflation fluid,
inserting through the applicator shaft a radiation source suitable for irradiating the vertebral tissue surrounding the cavity, the source being mounted at the end of a catheter or cable,
causing the source to emit therapeutic radiation to the cavity surfaces and into the diseased bone of the vertebra,
removing the radiation source and catheter or cable through the shaft and cannula,
draining the balloon of inflation fluid, and removing the balloon applicator through the cannula,
through the cannula, substantially filling the cavity with cement to stabilize the realigned position of the fractured vertebra, and
removing the cannula from the vertebra.

2. The method of claim 1, wherein the balloon of the balloon applicator includes an absorptive covering, and the method including delivering a radiosensitizing agent by perfusion through the absorptive covering to surfaces of the fracture zone, prior to emitting radiation to the cavity surfaces.

3. The method of claim 1, wherein the step of inserting the expandable brachytherapy balloon applicator immediately follows removal of a kyphoplasty balloon which has been used to expand the fracture zone.

4. A method for administering radiation therapy along with a spinal vertebral kyphoplasty, comprising:
in the kyphoplasty procedure, following placement of at least one cannula obliquely into the body of a collapsed or damaged vertebra and inflation of a fracture zone with a fluid delivered via the cannula, inserting through the cannula a radiation source suitable for irradiating the vertebral tissue surrounding the cavity, the source being mounted at the end of a catheter or cable,
causing the source to emit therapeutic radiation to the cavity surfaces and into the diseased bone of the vertebra,
removing the radiation source and catheter or cable through the shaft and cannula,
through the cannula, substantially filling the cavity with cement to stabilize the realigned position of the fractured vertebra, and
removing the cannula from the vertebra.

5. The method of claim 4, wherein the step of inserting the radiation source follows removal of a kyphoplasty balloon which has been used to expand the fracture zone.

6. The method of claim 4, wherein the step of inserting the radiation source follows use of a kyphoplasty balloon to expand the fracture zone by inflation using said fluid delivered via the cannula, and the step of inserting a radiation source comprises inserting the radiation source into the kyphoplasty balloon which has been left in place, such that the emission of the therapeutic radiation by the source is performed from within the balloon, and the method including removing the balloon prior to filling the cavity with cement.

7. The method of claim 6, wherein the fluid delivered into the balloon comprises a radiation attenuating fluid, through which the radiation source emits the therapeutic radiation.

8. The method of claim 4, wherein, prior to the step of inserting a radiation source, a kyphoplasty balloon which has been used to inflate and expand the fracture zone with said fluid is drained and removed, then a brachytherapy balloon applicator is inserted into the fracture zone through the cannula and inflated with an attenuating fluid using a shaft of the applicator extending through the cannula, so that the emission of radiation is from within the fluid-filled balloon applicator, and including draining and removing the balloon applicator prior to filling the cavity with cement.

9. The method of claim 8, wherein the balloon of the brachytherapy balloon applicator includes an absorptive covering, and the method including delivering a radiosensitizing agent by perfusion through the absorptive covering to surfaces of the fracture zone, prior to emitting radiation to the cavity surfaces.

10. The method of claim 4, further including, prior to inserting the radiation source through the cannula, applying a radiosensitizing agent to surfaces of the fracture zone of the vertebra.

11. The method of claim 10, wherein the application of the radiosensitizing is via swabbing, through the cannula.

12. The method of claim 10, wherein the application of the radiosensitizing agent is by delivering a liquid radiosensitizing agent through the cannula.
